(19) United States
(12) Patent Application Publication
(10) Pub. No.: US 2006/0095139 A1
(43) Pub. Date: May 4, 2006

(54) Composite prosthetic implant

(76) Inventors: Jean-Paul Gilbert Ricol, Vienne (FR); Richard Jean-Claude Guetty, Miribel (FR)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 10/518,199

(22) PCT Filed: Jun. 18, 2003
(86) PCT No.: PCT/FR03/01863

§ 371 Date: Aug. 9, 2005

(30) Foreign Application Priority Data
Jun. 18, 2002 (FR) 02/07698

(51) Int. Cl. A61F 2/02

(52) U.S. Cl. 623/23.72; 264/101; 427/2.24

(57) ABSTRACT

A composite prosthetic implant comprises a textile support of which at least a portion of the surface is covered by a lyophilisate of a biocompatible material, which comprises, as its main component, at least one of, and/or at least one of a derivative of, the following substances: hyaluronic acid, alginates, polypeptide, and polycaprolactone.

### TECHNICAL FIELD

This invention relates to the technical field of prosthetic implants used, notably, within the field of parietal surgery.

More specifically, this invention concerns a composite prosthetic implant comprising a textile support in conjunction with a biocompatible material, the said implant being intended for implantation by means of classic or coelioscopic surgery, for example in the treatment of hernias or eventrations.

This invention also concerns a process for producing a composite prosthetic implant in which a textile support. is impregnated with a solution of a first biocompatible material.

### PREVIOUS TECHNIQUE

It is already established practice to use prosthetic implants, for example, to strengthen and repair a damaged muscle wall.

One is thus already familiar with composite prosthetic implants comprising a textile network of which one of the sides is covered with a bioabsorbable film, the said film being superficially joined to the textile network by means of a biocompatible glue or by stitches or by means of direct impregnation.

The prosthetic implants of the type specified above are, however, of complex design which means that some of them can be susceptible to phenomena of delamination between the fabric and the bioabsorbable film.

By the same token, these implants are generally relatively heavy which obviously make them troublesome, in some cases leading to postoperative complications for the patient.

Moreover, the prosthetic implants of the previous type do not allow optimal cell rehabitation (recolonisation).

The complex and multi-layered structure of these implants also make it necessary to take special precautions so as to avoid any bacteriological development during manufacture, and this makes the manufacturing process complex and onerous, whereby there is also the risk that, because of the aforementioned drastic anti-bacteriological measures, of detracting from the active therapeutic principles which can be contained within the bioabsorbable film.

Besides, insertion and positioning of the implants of the prior art is generally delicate and awkward; it is, notably, very often difficult for the surgeon to guarantee precise positioning of the implant unless he uses staple-type fixing systems which are traumatising and onerous.

### DESCRIPTION OF THE INVENTION

The aims of the invention are consequently intended to propose a new composite prosthetic implant which does not have any of the disadvantages of the implants described above, and being of reduced mass.

Another aim of the invention is to propose a new composite prosthetic implant with improved mechanical properties.

Another aim of the invention is to propose a new composite prosthetic implant with improved cellular rehabitation properties.

Another aim of the invention is to propose a new composite prosthetic implant with improved hemostatic characteristics.

Another aim of the invention is to propose a new composite prosthetic implant which can offer bio-adhesive characteristics.

Another aim of the invention is to propose a new composite prosthetic implant of which the therapeutic properties are protected.

Another aim of the invention is to propose a new composite prosthetic implant which minimises the risk of postoperative infection.

Another aim of the invention is to propose a new process for producing a composite prosthetic implant which is particularly simple and easy to implement.

Another aim of the invention is to propose a new process for producing a composite prosthetic implant which is particularly fast to implement.

The aims of the invention are achieved with the help of a composite prosthetic implant comprising a textile support of which at least one portion of the surface is covered with a lyophilisate made from a biocompatible material, characterised in that the lyophilisate is a lyophilisate made from a biocompatible material which comprises, as the main component, one or several of the following substances, and/or one or several of the derivatives of the following substances:
hyaluronic acid,
alginates,
polypeptide,
polycaprolactone.

The aims of the invention are also achieved with the help of a process for manufacturing a composite prosthetic implant in which a textile support is impregnated with a solution of a first biocompatible material, the said process comprising a lyophilisation stage of the said first biocompatible material which takes place after the impregnation stage, characterised in that the first biocompatible material comprises, as a main component, one or several of the following substances, and/or one or several of the derivatives of the following substances:
hyaluronic acid,
alginates,
polypeptide,
polycaprolactone.

### BRIEF DESCRIPTION OF THE DRAWINGS

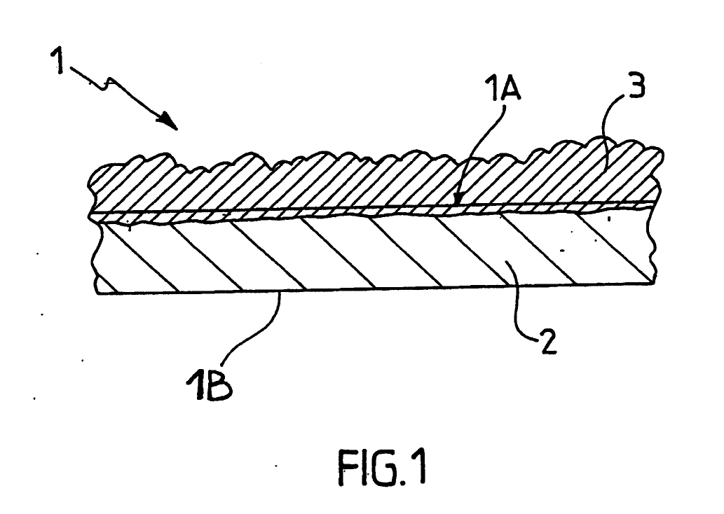

Other aims and advantages of the invention will become clearer when the attached description is read and with the help of the attached drawing, provided purely for illustration and information, in which FIG. 1 illustrates, by means of a cross section seen from the side, the schematic structure of a prosthetic implant in accordance with the invention.

### THE BEST WAY OF REALISING THE INVENTION

FIG. 1 shows a composite prosthetic implant in accordance with the invention, comprising a textile support 2, and intended to be implanted in the body of a patient, notably for the treatment of hernias or eventrations.

The term “implant” signifies here a prefabricated element intended to be introduced into the body of a patient. As such, an implant, in the sense of the invention, is clearly differentiated from creams or gels intended to be applied during surgical operations.

The term “composite” must be taken here in its most general sense, ie. it signifies an implant with a structure which is essentially heterogeneous.

In the sense of the invention, a textile support generally signifies a structural element involving fibres, and with a discontinuous character, contrary to a membrane, for example.

Advantageously, the said textile support 2 comprises a top layer which is bi-dimensional or tri-dimensional in structure.

This textile layer can be of any type, and notably non-woven, woven or interlaced.

Preferably, this textile layer is a chain-knitted layer.

The textile support 2 can be made from threads of any type, and notably biocompatible polymer threads, resorptive or not.

Preferably, the textile support 2 will be biocompatible but not resorptive.

Advantageously, the textile support 2 is made from polyester or polypropylene threads.

These threads can be single-stranded or multi-stranded.

In a preferred variation, a knitted fabric based on polyester multi-stranded threads will be used.

In accordance with the invention, the said textile support 2 is associated with a biocompatible material.

“Biocompatible material” signifies here any implantable biomaterial, bioabsorbable or not.

In accordance with the invention, the said biocompatible material comprises as its main component one or several of the following substances, and/or one or several of the derivatives of the following substances:
polysaccharide, and preferably: chitosan, hyaluronic acid, alginates,
collagen, bovine or marine, native or not,
polypeptide, and preferably: polypeptide of the polyalpha amino acid type, and more preferably a copolymer of leucine and methyl glutamate,
polycaprolactone.

In accordance with one essential characteristic of the invention, at least one portion of the surface 1A of the textile support 2 is covered by a lyophilisate 3 of the biocompatible material.

The lyophilisation of the biocompatible material makes it possible to obtain a lyophilisate 3 which is in the form of a porous material which lends itself particularly well to cellular rehabitation.

In addition, the porous character of this material means that it is a particularly light material such that a film of classic biocompatible material, of the membrane type, is perceptibly ten times heavier than a lyophilisate of the same material, covering an equal surface.

The use of a lyophilisate for a prosthetic implant thus makes is possible to obtain a particularly light prosthesis which is therefore easier for the patient to tolerate.

The material (lyophilisate) obtained from the lyophilisation also presents a spongy character which gives it good hemostatic properties and favours a possible biological sticking of the implant to a biological tissue.

The lyophilisation of the biocompatible material also makes it possible to conserve and protect the qualities of the active principles contained in the biocompatible material, and notably the possible scarring and antibacterial qualities.

Advantageously, the lyophilisate 3 is a lyophilisate made from a biocompatible material which comprises, as its main component, one or several of the following substances, and/or one or several of the derivatives of the following substances:
hyaluronic acid,
alginates,
polypeptide,
polycaprolactone.

In other words, the lyophilisate 3 can comprise any one of the four substances (or one of its derivatives) specified above, or a mixture of two, three or four of these substances (or of their derivatives). A mixture of derivatives and pure substances is, of course, equally possible.

More preferably, the lyophilisate 3 is a lyophilisate of hyaluronic acid, and notably of hyaluronic acid with a molecular mass of between 800,000 and 2,000,000 daltons, and more preferably, of between 1,200,000 and 1,500,000 daltons.

Preferably, the lyophilisate 3 is joined closely with the textile support 2 and penetrates into the thickness of the latter, as represented schematically in FIG. 1.

The textile support 2 and the lyophilisate 3 thus form a coherent material, the components of which (textile support and lyophilisate) are quite inseparable.

This type of integrated structure is especially interesting from the point of view of the mechanical properties of the prosthetic implant in accordance with the invention because it makes it possible to reduce the risk of delamination between the textile support and the biocompatible material.

The prosthetic implant 1 in accordance with the invention is thus preferably in the form of a textile substratum 2 which creates a first layer, this first layer 2 comprising a first and an opposite second side 1A, 1B. The first side 1A of the first layer 2 is itself covered, preferably in its entirety, by a second layer 3 formed by the lyophilisate 3. In another version, the two sides 1A, 1B of the substratum 2 are covered respectively by a second and a third layer of lyophilisate, the said two layers of lyophilisate being able to be of identical or different types in terms, notably, of thickness or composition.

The prosthetic implant 1 in accordance with the invention is thus in a complex, multi-layered form made up of a series of superimposed layers 2, 3, and all joined together, preferably over the whole contact surface.

In the case where the lyophilisate 3 is made from hyaluronic acid, this lyophilisate 3 is in the form of a layer of foam which is relatively dry and non-sticky to the touch. When this layer of foam is moistened with liquid, the said layer then becomes sticky, and this allows the surgeon, notably in the case of cures for hernias or eventrations, to stick the implant to the parietal tissues without using invasive or traumatising methods such as staples or sutures. This sticky characteristic which can be activated is particularly effective in the case of a lyophilisate 3 based exclusively on hyaluronic acid. This characteristic is no less present in cases where other materials are used, for example alginate of sodium or chitosan.

Advantageously, a lyophilisate 3 with bioresorptive characteristics will be used.

The invention equally relates to a process for the manufacture of a composite prosthetic implant in accordance with the invention.

In this process, a textile support 2 is impregnated with a solution of a first biocompatible material. This impregnation can, for example, be carried out using soaking.

The term “solution” signifies a substance, the characteristics of which with regard to viscosity and wet-ability are compatible with an operation of the coating or impregnation type, unlike a substance in solid state.

In accordance with one important characteristic of the manufacture process in accordance with the invention, the said process comprises a lyophilisation stage for the said first biocompatible material, the said lyophilisation stage taking place after the aforementioned impregnation stage.

The process in accordance with the invention thus makes is possible to obtain a lyophilisate 3 on the surface of the textile support 2, the said lyophilisate 3 being substantially made as one piece with the impregnated textile support.

In accordance with the invention, the first biocompatible material comprises, as its main component, one or several of the following substances, and/or one or several of the derivatives of the following substances:
hyaluronic acid,
alginates,
polypeptide,
polycaprolactone.

It is particularly interesting to note that the lyophilisation can be schematically assimilated to a pre-sterilisation, in the sense where it minimises bacteriological development, and notably, for example, the development of salmonella.

This process of manufacture in accordance with the invention is thus particularly safe from the point of view of bacteriological risk.

Advantageously, the process in accordance with the invention comprises, subsequently to the aforementioned impregnation stage and prior to the lyophilisation stage, a pouring stage in which one pours a solution of a second biocompatible material over the pre-impregnated textile support. The second biocompatible material preferably comprises, as its main component, one or several of the following substances, and/or one or several of the derivatives of the following substances:
hyaluronic acid,
alginates,
polypeptide,
polycaprolactone.

In one particular form, the second material is similar to the first material.

The solution of the second biocompatible material then also undergoes lyophilisation during the lyophilisation stage.

Advantageously, the process in accordance with the invention comprises, subsequently to the impregnation stage and prior to the lyophilisation stage, a coating stage in which the impregnated textile support is coated with a layer of a solution of a third biocompatible material.

The third biocompatible material preferably comprises, as its main component, one or several of the following substances, and/or one or several of the derivatives of the following substances:
hyaluronic acid,
alginates,
polypeptide,
polycaprolactone.

In one embodiment in particular, the third material is similar to the first material and/or to the second material.

The solution of the third biocompatible material then also undergoes lyophilisation during the lyophilisation stage.

The aforementioned pouring and coating stages follow a similar procedure, the difference being that one will opt for pouring if dealing with a solution with a low level viscosity, and coating if dealing with a solution which has a higher level of viscosity.

Advantageously, the process in accordance with the invention comprises a spreading-out stage during which one spreads out on the lyophilisation tray used during the lyophilisation stage, a layer of the solution of a fourth biocompatible material, then one places against this layer the textile support 2 impregnated with the solution of the first biocompatible material.

The fourth biocompatible material preferably comprises, as its main component, one or several of the following substances, and/or one or several of the derivatives of the following substances:
hyaluronic acid,
alginates,
polypeptide,
polycaprolactone.

In one particular embodiment, the fourth biocompatible material is similar to the first material and/or the second material and/or the third material.

The solution of the fourth biocompatible material then also undergoes lyophilisation during the lyophilisation stage.

Advantageously, the manufacturing process in accordance with the invention comprises a drying stage for the impregnated textile support, the said drying stage taking place following the impregnation stage.

It is therefore clear that the invention generally relates to the application of a lyophilisate 3 to the surface of a textile support for the purpose of making surgical prostheses, no matter which of the numerous versions of the concept is concerned, and which the specialist will be able to grasp by reading this description.

The prosthetic implant in accordance with the invention consequently has improved mechanical properties from the point of view of its anchoring, its resistance and its flexibility—all of which are particularly desirable for coelioscopic applications (using a trocar). This flexibility results on the one hand, notably, from the spongy character of the lyophilisate 3 which is not intrinsically fragile in character and is less prone to breakage or splitting than the previous types of film, and on the other hand from close joining of the lyophilisate 3 and the textile support 2.

Preferably, the implant 1 in accordance with the invention is sterilised, for example by gamma rays.

Some examples of prosthetic implants in accordance with the invention will now be described.

### EXAMPLE 1

A solution of 1% hyaluronic acid with a molecular mass of approx. 800,000 daltons is prepared by means of hydration of sodium hyaluronate in sterile water for injection (purified water).

The solution obtained in this way is poured into a beaker into which is then placed a prosthetic fabric made from multi-stranded, knitted polyester threads (PES).

The fabric is left in the beaker for fifteen to thirty minutes so that the fibres are well impregnated with hyaluronic acid.

The fabric impregnated in this way is then placed on the tray of a lyophilisator, and a small amount of the hyaluronic acid solution is poured onto the impregnated fabric.

The tray is then placed in congelation at −40° C. for three hours. Sublimation then takes place at from −40° C. to +30° C. up to 0.25 millibars for 18½ hours, and then desorption at 30° C. and 0.03 millibars for 7 hours. In this way one obtains a prosthetic implant made from a fabric, the pores of which are blocked by the lyophilisate.

### EXAMPLE 2

A first homogeneous solution is prepared of 1% hyaluronic acid with a molecular mass equal to approx. 800,000 daltons by means of hydration of sodium halyuronate in sterile water for injection.

A second solution is then prepared of 2% hyaluronic acid with a molecular mass equal to approx. 800,000 daltons by means of hydration of sodium halyuronate in sterile water for injection.

The first solution is poured into a beaker into which one then places a fabric made from multi-stranded knitted polyester threads (PES).

The fabric is left in the beaker for 15 to 30 minutes so that the fibres are well impregnated with hyaluronic acid.

Using a spatula, a layer of the second solution is then spread out over the tray of a lyophilisator, in such a way that this layer is approx. 3 mm thick. The pre-impregnated and drained fabric is then placed on this layer of the second solution. A second layer is then spread out, similarly to the first layer, over the fabric and on the opposite side to that which is in contact with the first layer.

The tray is then placed in congelation at −40° C. for 3 hours. Sublimation then takes place at from −40° C. to +30° C. up to 0.25 millibars for 18½ hours, and then desorption at 30° C. and 0.03 millibars for 7 hours.

In this way one obtains a prosthesis coated on both sides with lyophilisate. The surface mass of the lyophilisate of hyaluronic acid is approx. 1 g per 100 cm².

As a variation, the fabric impregnated with the first solution is left to dry before coating it with the second solution.

### EXAMPLE 3

A first homogeneous solution is prepared of 0.7% hyaluronic acid with a molecular mass equal to approx. 1,570,000 daltons by means of hydration of sodium hyaluronate in sterile water for injection. A second homogeneous solution is prepared of 1.5% hyaluronic acid with a molecular mass equal to approx. 1,570,000 daltons by means of hydration of sodium hyaluronate in sterile water for injection.

The first solution is poured into a beaker into which one then places a prosthetic fabric made from multi-strand, knitted polyester threads (PES). The fabric is kept in the beaker for 15 to 30 minutes so that the fibres are well impregnated with hyaluronic acid.

The impregnated fabric, which has been previously drained, is placed on the tray of a lyophilisator. Using a spatula, a layer of the second solution, approx. 3 mm thick, is then spread out.

The tray is then placed for an hour at −80° C. and then for 2 hours at −40° C. Sublimation then takes place at from −40° C. to +50° C. up to 0.25 millibars for 12 hours, and then desorption at 60° C. and 0.03 millibars for 7 hours.

An implant is obtained of which one of its sides is coated with lyophilised hyaluronic acid. The surface mass of the hyaluronic acid lyophilisate is equal to approx. 0.5 g per 100 cm².

As a variation, just one part of one of the sides of the fabric is coated with a layer approx. 3 mm thick of the second solution, the rest of this side being masked by a silicone template. The template is taken off between the first stage of congelation at −80° C. for 1 hour and the second stage of congelation at −40° C. for 2 hours. The lyophilisation cycle already described is then realised. On thus obtains, in accordance with this variation, an implant of which just one part of one of its sides is covered with lyophilised hyaluronic acid.

### EXAMPLE 4

A solution of 1% sodium alginate is prepared in water for injection (purified water).

A fabric made principally from multi-strand knitted polyester is impregnated by the above solution, and the fabric is then placed on the tray of a lyophilisator and coated with the solution produced as above.

The tray is then placed in congelation at −40° C. for three hours.

Sublimation then takes place at from −40° C. to +30° C. up to 0.25 millibars for 18½ hours, and then desorption at 30° C. and 0.03 millibars for 7 hours. A tissue implant is obtained of which the pores are blocked by lyophilisate.

### EXAMPLE 5

A solution is made of 1% chitosan in water for injection with 30 drops of acetic acid.

A fabric made from multi-strand knitted polyester threads (PES) is impregnated with this solution. The impregnated fabric is placed on the tray of a lyophilisator, and it is coated with the above solution. The tray is then placed in congelation a −40° C. for 3 hours, and then sublimation takes place at from −40° C. to +30° C. up to 0.25 millibars for 18½ hours, and then finally desorption at 30° C. and 0.03 millibars for 7 hours. A fabric is obtained, the pores of which are blocked by the lyophilisate. The lyophilisate is quite yellow in colour, and its anchoring to the fabric is less than that of the lyophilisates of hyaluronic acid from the previous examples.

### POSSIBILITIES FOR INDUSTRIAL APPLICATION

The invention has its industrial application in the manufacture and use of surgical implants.

1. A composite prosthetic implant comprising:
a textile support of which at least a portion of the surface is covered by a lyophilisate of a biocompatible material, which comprises, as its main component, at least one of, and/or at least one of a derivative of, the following substances:
hyaluronic acid,
alginates,
polypeptide, and
polycaprolactone.

2. The prosthetic implant of claim 1, wherein the lyophilisate is a lyophilisate of hyaluronic acid with a molecular mass of between 800,000 and 2,000,000 daltons.

3. The prosthetic implant of claim 1, wherein said textile support comprises a top layer of bidimensional or tridimensional structure, the top layer selected from the group consisting of:
a non-woven layer,
a woven layer,
a knitted layer, and
an interlaced layer.

4. The prosthetic implant of claim 1, wherein said textile support is obtained from threads chosen from the group consisting of:
single-strand or multi-strand polyester threads and
single-strand or multi-strand polypropylene threads.

5. (canceled)

6. A process for the manufacture of a composite prosthetic implant comprising:
an impregnation stage comprising impregnating a textile support with a solution of a first biocompatible material, the first biocompatible material comprising, as its main component, at least one of and/or at least one of a derivative of, the following substances:
hyaluronic acid,
alginates,
polypeptide, and
polycaprolactone;
wherein the process further comprises a lyophilisation stage of the said first biocompatible material. the lyophilisation stage occurring after the impregnation stage.

7. The process of claim 6, further comprising:
a pouring stage, subsequent to the impregnation stage and prior to the lyophilisation stage, in which a solution of a second biocompatible material is poured onto the impregnated textile support.

8. The process of claim 6, further comprising:
a coating stage, subsequent to the impregnation stage and prior to the lyophilisation stage, in which the impregnated textile support is coated with a solution of a third biocompatible material.

9. The process of claim 6. further comprising:
a spreading-out stage, in which a layer of the solution of a fourth biocompatible material is spread out on the a tray of the lyophilisator used in a lyophilisation stage, and the textile support impregnated with the solution of the first biocompatible material is then placed against the layer.

10. The process of claim 6, further comprising:
a drying stage, following the impregnation stage, for the impregnated textile support.

11. A method comprising:
covering a prosthetic implant with a lyophilisate layer; and
sticking the lyophilisate layer to biological tissue.

12. The method of claim 11, wherein the method further comprises:
surgically implantating the prosthetic implant for the treatment of a hernia or eventration.

13. The prosthetic implant of claim 1, wherein the lyophilisate is a lyophilisate of hyaluronic acid with a molecular mass of between 1,200,000 and 1,500,000 daltons.

14. The prosthetic implant of claim 2, wherein the lyophilisate is a lyophilisate of hyaluronic acid with a molecular mass of between 1,200,000 and 1,500,000 daltons.

15. The prosthetic implant of claim 2, wherein said textile support comprises a top layer of bidimensional or tridimensional structure, the top layer selected from the group consisting of:
a non-woven layer,
a woven layer,
a knitted layer, and
an interlaced layer.

16. The prosthetic implant of claim 2, wherein said textile support is obtained from threads chosen from the group consisting of:
single-strand or multi-strand polyester threads and
single-strand or multi-strand polypropylene threads.

17. The prosthetic implant of claim 3, wherein said textile support is obtained from threads chosen from the group consisting of:
single-strand or multi-strand polyester threads and
single-strand or multi-strand polypropylene threads.

18. The process of claim 7, further comprising:
a spreading-out stage, in which a layer of the solution of a fourth biocompatible material is spread out on a tray of a lyophilisator used in the lyophilisation stage, and the textile support impregnated with the solution of the first biocompatible material is then placed against the layer.

19. The process of claim 8, further comprising:
a spreading-out stage, in which a layer of the solution of a fourth biocompatible material is spread out on a tray of a lyophilisator used in the lyophilisation stage, and the textile support impregnated with the solution of the first biocompatible material is then placed against the layer.
